(19) United States
(12) Patent Application Publication
(10) Pub. No.: US 2026/0284371 A1
(43) Pub. Date: Sep. 24, 2026

(54) METHOD AND APPARATUS FOR EPIDERMAL DELIVERY OF POWDERED MEDICAMENTS

(71) Applicant: PARTICLE VACCINE CANADA LTD., Ottawa (CA)

(72) Inventors: Christopher RODRIGUEZ, Carlsbad, CA (US); Cameron FALKENBURG, San Diego, CA (US); Leonard ANGKA, Gloucester (CA); John Frederick CORNHILL, Fort Lauderdale, FL (US)

(73) Assignee: PARTICLE VACCINE CANADA LTD., Ottawa, ON (CA)

(21) Appl. No.: 19/687,980

(22) Filed: May 26, 2026

Related U.S. Application Data
(63) Continuation of application No. 18/243,404, filed on Sep. 7, 2023.
(60) Provisional application No. 63/404,382, filed on Sep. 7, 2022.

(51) Int. Cl. A61M 37/00; A61M 5/20; A61M 5/30; A61M 35/00

(52) U.S. Cl. CPC A61M 37/00; A61M 5/2053; A61M 5/30; A61M 35/003; A61M 2202/064; A61M 2205/8218; A61M 2205/8225

(57) ABSTRACT

Apparatus for transdermal delivery of a powdered agent to a patient, the apparatus comprising a fluid source comprising a fluid; a nozzle extending distally from the fluid source, the nozzle comprising a proximal end, a distal end and a lumen extending from the proximal end to the distal end; a blister containing a powdered agent disposed within the lumen of the nozzle; and an actuation element for releasing the fluid from the fluid source, wherein the actuation element causes the released fluid to be propelled through the blister with sufficient pressure to entrain the powdered agent into the released fluid and move the entrained powdered agent through the lumen of the nozzle and out the distal end of the nozzle.

### CROSS REFERENCE TO RELATED APPLICATION

The present application is a continuation of U.S. patent application Ser. No. 18/243,404, entitled “METHOD AND APPARATUS FOR EPIDERMAL DELIVERY OF POWDERED MEDICAMENTS” filed on Sep. 7, 2023, which claims priority to U.S. Provisional Patent Application No. 63/404,382, entitled “METHOD AND APPARATUS FOR EPIDERMAL DELIVERY OF POWDERED MEDICAMENTS”, filed on filed Sep. 7, 2022, the contents of which are incorporated herein by reference.

### FIELD OF THE INVENTION

This invention relates to methods and apparatus for the epidermal administration of medicaments in general, and more particularly to methods and apparatus for the epidermal administration of powdered medicaments using a compressed gas.

### BACKGROUND OF THE INVENTION

The COVID-19 global pandemic has shown that our ability to manufacture, transport, and administer life-saving vaccines to the population in an effective and timely manner is not fit for purpose. Vaccine shortages forced governments all over the world to ration their vaccine supplies which delayed herd immunity and prolonged the city-wide lockdowns.

The manufacturing and distribution of vaccines for a global population requires many resources and is limited by the amount of active ingredient needed to generate a biological immune response. In order to reduce the amount of active ingredient needed to generate a biological immune response, it would be beneficial if the active ingredients (e.g., antigens) that are in vaccines were delivered directly to regions in the body that are rich in immune cells (i.e., “antigen presenting cells”). The epidermis (i.e., the outer layer of the skin) contains a dense network of antigen presenting cells known as Langerhans cells (FIG. 1), and these immune cells are very effective at generating potent immune responses. Targeting the Langerhans cells can be an effective approach that results in substantial dose sparing, which reduces the amount of active ingredient required for a full immune effect (e.g., the amount of active ingredient required for a protective immune response can be reduced to approximately 1/10th of the amount of active ingredient typically required). Therefore, delivery of medicaments (e.g., drugs, vaccines, proteins, peptides, oligonucleotides, biologics, etc.) directly to Langerhans cells (or other immune cells) offers a promising alternative approach for generating a protective immune response while also reducing the amount of active ingredient required to generate the protective immune response.

However, delivery of medicaments is very challenging to accomplish with conventional liquid vaccines delivered by needle/syringe (e.g., intramuscular injection), and is not preferred during a pandemic, because delivery of conventional liquid vaccines via needle/syringe generally requires a high degree of technical skill, and highly-trained healthcare workers are limited, especially in remote and rural communities where highly-trained health care workers and facilities are few and far between. Therefore, it is desirable that the delivery of vaccines to the high density region of Langerhans cells be administered by anyone with very little training and skill, whereby to be suitable for rapid pandemic response.

In addition, the Centers for Disease Control (CDC) estimates that as many as 2 in 3 children (and 1 in 4 adults) suffer from trypanophobia (i.e., a fear of needles). Fear of needles is a large barrier for most vaccination schedules, especially in children, and burdens the healthcare system in time, effort, and importantly—vaccine compliance. A transdermal needle-free delivery device can minimize both the physical and psychological trauma to the patient receiving the medication. In addition, by avoiding the need to pierce the skin of a patient with a sterile element (e.g., a needle), transdermal needle-free delivery of medicaments would completely eliminate the issues of needle-stick injury and minimize the possibilities of infection resulting from the accidental reuse of discarded or previously used needles.

One way to provide needle-free delivery of a vaccine or other medicament is by providing the vaccine (or other medicament) in the form of a powder, entraining the powder in a jet of gas, and directing the jet of gas and entrained powdered agents toward the skin of the patient. It will be appreciated that, as used herein, the terms “powdered medicaments” or “powdered agents” are intended to encompass particle or powder vaccines in addition to such other medicaments or agents as are typically present in a vaccine or other medicament (e.g., adjuvants to increase the immune response to the vaccine, preservatives, secondary medicaments, etc.). By providing the vaccine (or other medicament) in the form of a powder, and entraining the powder in a jet of gas, the powder can be accelerated inside the jet of gas such that the momentum of the entrained particles is sufficient to travel to the desired depth to achieve the desired therapeutic purpose. To this end, it is important to note that penetration depth of the entrained powder has been found to be a function of particle size, particle density and pressure of the gas jet (i.e., momentum, which is the product of the particle's mass times the velocity of the particle imparted by the gas jet). Furthermore, humidity, temperature, age of the patient and ethnicity of the patient can all affect the resilience of the skin. Therefore, successful needle-free epidermal delivery requires enough penetration force to breach the stratum corneum (i.e., the exterior of the skin), and deliver the vaccine volume to the high density region of Langerhans cells. Preferably, the pressure of the gas should be adjustable so that any particle vaccine can be targeted to the dense network of Langerhans cells of all patients under any environmental condition.

While entraining powdered medicaments in a jet of gas has been proven in laboratory experiments as a viable route of delivering powdered medicaments, numerous engineering challenges have remained unsolved.

By way of example but not limitation, current needle-free epidermal delivery devices are limited to one dose of medicament per device which leads to excess amounts of waste and cost. Therefore, a single device that could be used to deliver multiple sterile, individually packaged doses of powdered medicament to multiple patients before the device is discarded would be more environmentally responsible and at the same time decrease the cost of mass vaccination programs.

By way of further example but not limitation, another engineering challenge relates to maintaining the tip of the device used to effect delivery of the powdered agent in contact with the skin of the patient for a long enough period of time to result in an effective delivery of the medicament (e.g., a particle vaccine). Specifically, as the jet of gas encounters the surface of the skin in the confines of a tube of a delivery device pressed against the skin, the pressure change can cause the delivery tube to move out of position, resulting in an ineffective delivery due to misalignment of the device against the skin of the patient. Thus, it would be beneficial to provide a device which reduces the amount of misalignment between the delivery device and the skin of the patient so that the medicament (e.g., a particle vaccine) can be delivered directly to the epidermis. It would also be beneficial for the device to trap any excess particle vaccine within the delivery device (e.g., in a disposable and biodegradable “nozzle” which also houses a sterile particle vaccine dose).

Thus there is a need for a new and improved method and apparatus for effecting the epidermal delivery of powdered agents or medicaments (e.g., powdered medicaments, particle vaccines, etc.) that addresses the issues of vaccine shortages through dose sparing, has a lower skill requisite for communities with less access to healthcare professionals, eliminates needles, has an adjustable gas jet pressure to account for the different pressures required to deliver different types of powdered vaccines in different environmental conditions and to different tissue densities, can be used to deliver multiple doses and maintains effective contact with the surface of the skin during the delivery process, whereby to provide a new and improved way to effectively dose and administer powdered agents (e.g., powdered medicaments, particle vaccines, etc.) to the desired penetration depth (e.g., a depth comprising a high-density region of Langerhans cells).

### SUMMARY OF THE INVENTION

The present invention comprises the provision and use of a new and improved method and apparatus for effecting the epidermal delivery of powdered agents or medicaments (e.g., powdered medicaments, particle vaccines, etc.) which (i) enables multiple metered doses of a vaccine/therapeutic medicament to be delivered through the skin of a patient within the depth boundaries of the epidermis where the Langerhans cells reside for effective vaccination, (ii) uses a novel needle-free delivery system that uses compressed gas to entrain a powdered agent(s) (e.g., powdered medicaments) that is disposed in a single disposable and biodegradable nozzle/delivery tube into a jet of gas for delivery to a patient, (iii) contains an adjustable pressure metering system to permit clinicians and contract manufacturers the ability to set the desired pressure for delivering a particular powdered agent (e.g., a particle-based medicament) to a patient, and (iv) addresses the issues of nozzle/delivery tube movement during delivery and the potential for the escape of powdered agent (e.g., powdered medicament) into the ambient air in the area of the delivery.

More particularly, the present invention comprises a novel handheld epidermal delivery device having a single reservoir of compressed gas and a disposable nozzle/delivery tube which enables the device to be used to reliably and repeatedly dispense a jet of gas for entraining and delivering multiple doses of sterile, individually packaged doses of powdered medicament through the skin of a patient. The reusable device of the present invention enables a single device to be used to deliver more than a single dose before the device is discarded, which is more environmentally responsible and at the same time decreases the cost of mass vaccination programs.

The disposable nozzle or delivery tube of the present invention preferably comprises novel features to reduce the amount of misalignment between the distal end of the delivery tube and the skin of the patient so that the medicament (e.g., a particle vaccine) can be delivered directly to the epidermis and any excess particle vaccine is trapped within a disposable and biodegradable “nozzle” that houses the sterile particle vaccine dose.

In one preferred form of the present invention, there is provided apparatus for transdermal delivery of a powdered agent to a patient, the apparatus comprising:
a fluid source comprising a fluid;
a nozzle extending distally from the fluid source, the nozzle comprising a proximal end, a distal end and a lumen extending from the proximal end to the distal end;
a blister containing a powdered agent disposed within the lumen of the nozzle; and
an actuation element for releasing the fluid from the fluid source, wherein the actuation element causes the released fluid to be propelled through the blister with sufficient pressure to entrain the powdered agent into the released fluid and move the entrained powdered agent through the lumen of the nozzle and out the distal end of the nozzle.

In another preferred form of the present invention, there is provided a method for transdermally delivering a powdered agent to a patient, the method comprising:
providing apparatus comprising:
a fluid source comprising a fluid;
a nozzle extending distally from the fluid source, the nozzle comprising a proximal end, a distal end and a lumen extending from the proximal end to the distal end; and
a blister containing a powdered agent disposed within the lumen of the nozzle;
positioning the distal end of the nozzle against the skin of the patient;
releasing the fluid from the fluid source, whereby to cause the fluid to be propelled through the blister with sufficient pressure to entrain the powdered agent into the released fluid and move the entrained powdered agent through the lumen of the nozzle, out the distal end of the nozzle and through the skin of the patient.

In another preferred form of the present invention, there is provided a method for transdermally delivering a powdered agent to a patient, the method comprising:
providing a delivery device comprising a proximal end, a distal end and a lumen extending from the proximal end to the distal end, wherein a blade is disposed in the lumen of the delivery device;
positioning a deformable cavity containing a powdered agent proximal to the blade;
positioning the distal end of the delivery device against the skin of a patient;
causing a stream of fluid to pass through the deformable cavity with sufficient force to cause the deformable cavity to move distally towards the blade, pierce the deformable cavity and release the powdered agent into the stream of fluid for passage out of the distal end of the lumen and into the skin of the patient.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts and further wherein:

FIG. 1 is a schematic view showing aspects of an exemplary epidermis of a human patient;

FIGS. 2-5 and 6A are schematic views showing a novel handheld epidermal delivery device formed in accordance with the present invention;

FIG. 6B is a process flow diagram showing further aspects of the novel handheld epidermal delivery device of FIGS. 2-5 and 6A;

FIGS. 7, 8 and 9A-9C are schematic views showing a novel distal delivery tube of the novel handheld epidermal delivery device of FIGS. 2-5 and 6A;

FIGS. 10A-10G are schematic views showing exemplary blade elements formed in accordance with the present invention;

FIGS. 11A, 11B, 12A, 12B, 13A and 13B are schematic views showing aspects of a novel blister formed in accordance with the present invention;

FIGS. 14A-14C are schematic views showing a novel blister formed in accordance with the present invention disposed within a novel distal delivery tube formed in accordance with the present invention;

FIGS. 15A-15D and 16A-16C are schematic views showing how the novel blister of FIGS. 11A, 11B, 12A, 12B, 13A and 13B may be deformed and/or pierced by a jet of gas;

FIGS. 17A-17C are schematic views showing how a jet of gas can be used to pierce a novel blister of FIGS. 11A, 11B, 12A, 12B, 13A and 13B so as to permit the contents of the blister to be passed through the distal delivery tube of the novel handheld epidermal delivery device of FIGS. 2-5 and 6A so as to cause the contents of the blister to be entrained in the jet of gas in order to penetrate the skin of a patient;

FIGS. 17D-17H are schematic views showing alternative embodiments of the novel distal delivery tube of FIGS. 7, 8 and 9A-9C formed in accordance with the present invention;

FIGS. 18A and 18B are schematic views showing a novel safety system formed in accordance with the present invention;

FIGS. 19A and 19B are schematic views showing a novel connector for connecting a distal delivery tube to the novel handheld epidermal delivery device of the present invention;

FIGS. 20A and 20B are schematic views showing another novel connector for connecting a distal delivery tube to the novel handheld epidermal delivery device of the present invention;

FIG. 21 is a schematic view showing still another novel connector for connecting a distal delivery tube to the novel handheld epidermal delivery device of the present invention;

FIGS. 21A-21E are schematic views showing yet another novel connector for connecting a distal delivery tube to the novel handheld epidermal delivery device of the present invention;

FIGS. 22A, 22B, 23A, 23B, 24A and 24B are schematic views showing alternative forms of a distal delivery tube formed in accordance with the present invention; and

FIG. 25 is a schematic view showing a novel suction cup distal interface formed in accordance with the present invention.

### DETAILED DESCRIPTION OF THE PREFERRED EMBODIMENTS

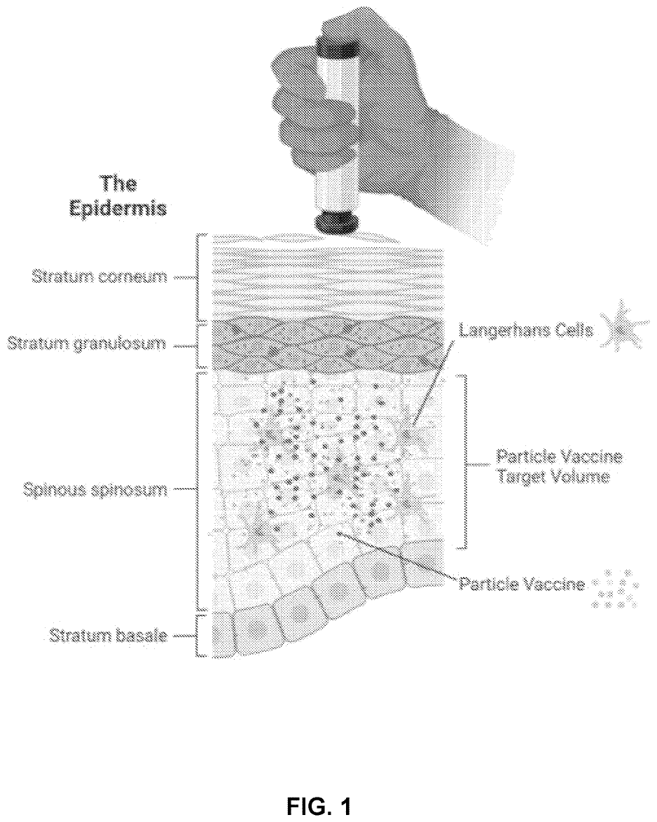

The present invention comprises the provision and use of a new and improved method and apparatus for effecting the delivery of powdered agents through the epidermis to a high density area of Langerhans cells (FIG. 1), as discussed above. As used herein, the terms “powdered agent” and/or “powdered medicament” are intended to encompass substantially any substance that may be provided in powdered or particle form including, but not limited to, particle vaccine and/or adjuvants and/or preservatives and/or secondary medicaments and/or agents.

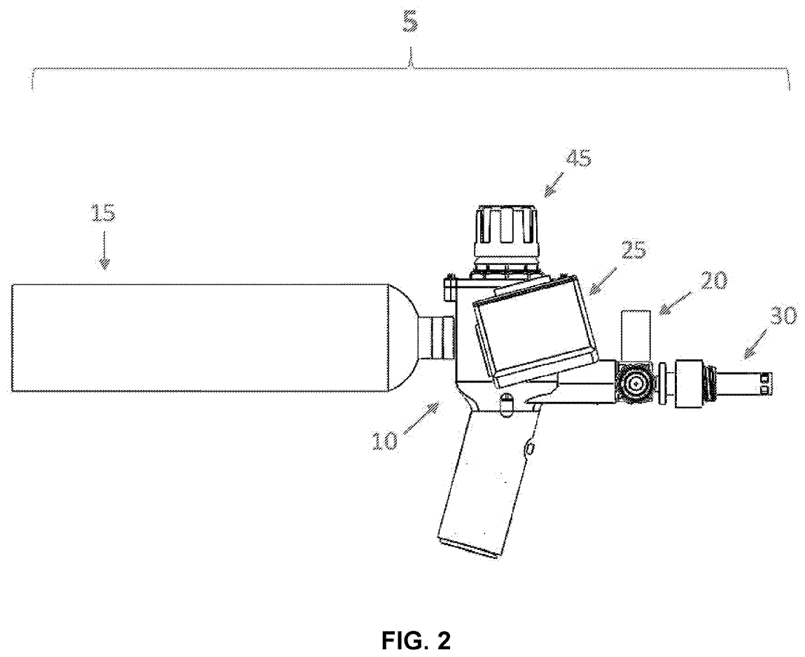

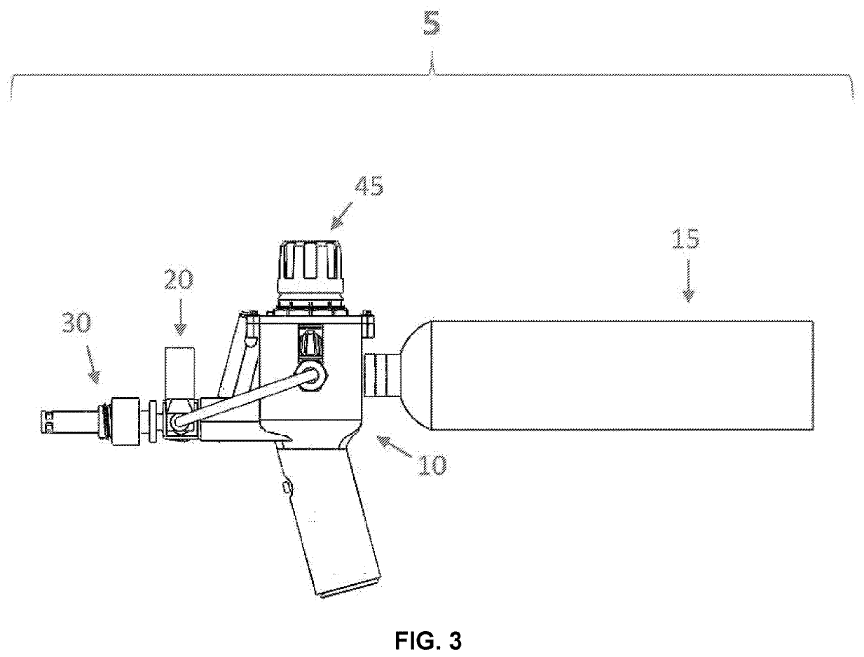

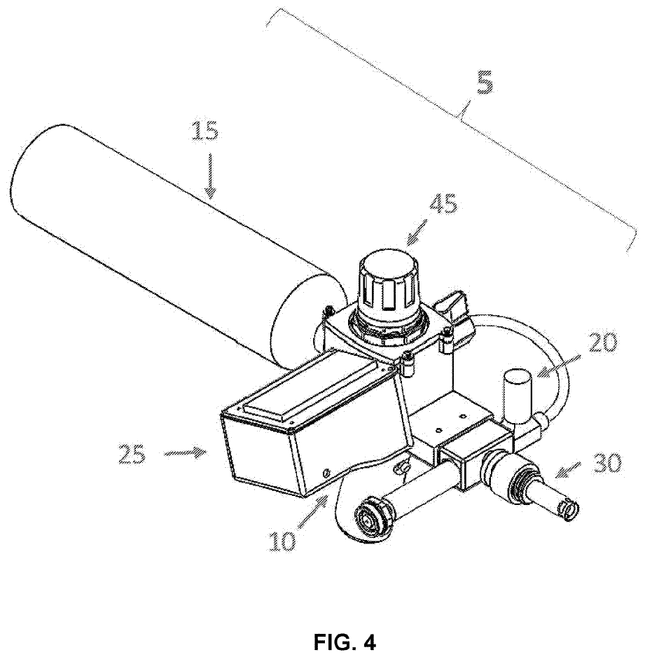

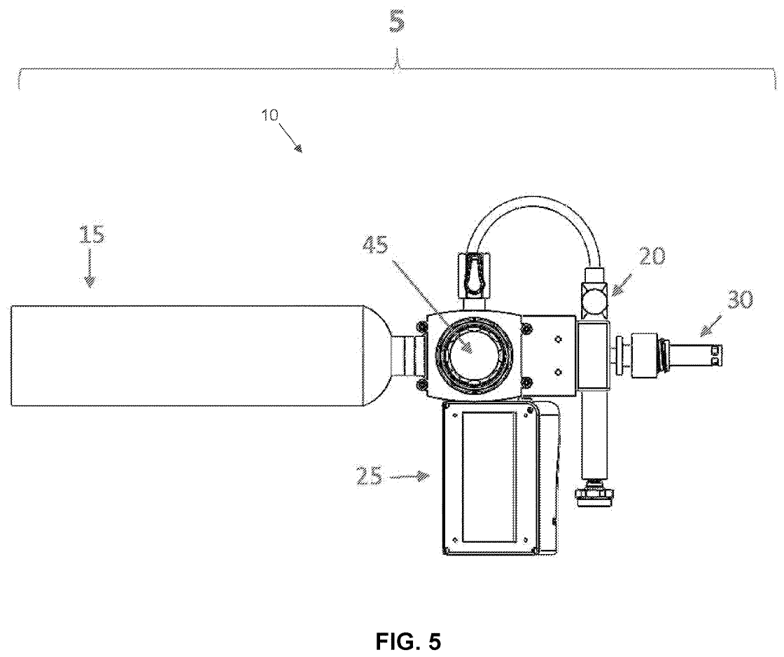

In FIGS. 2-5, there is shown a novel handheld epidermal delivery device 5 formed in accordance with the present invention. Epidermal delivery device 5 generally comprises a body 10, a gas storage tank 15, gas metering assembly 20, an electronics box 25 and a nozzle 30.

### The Body

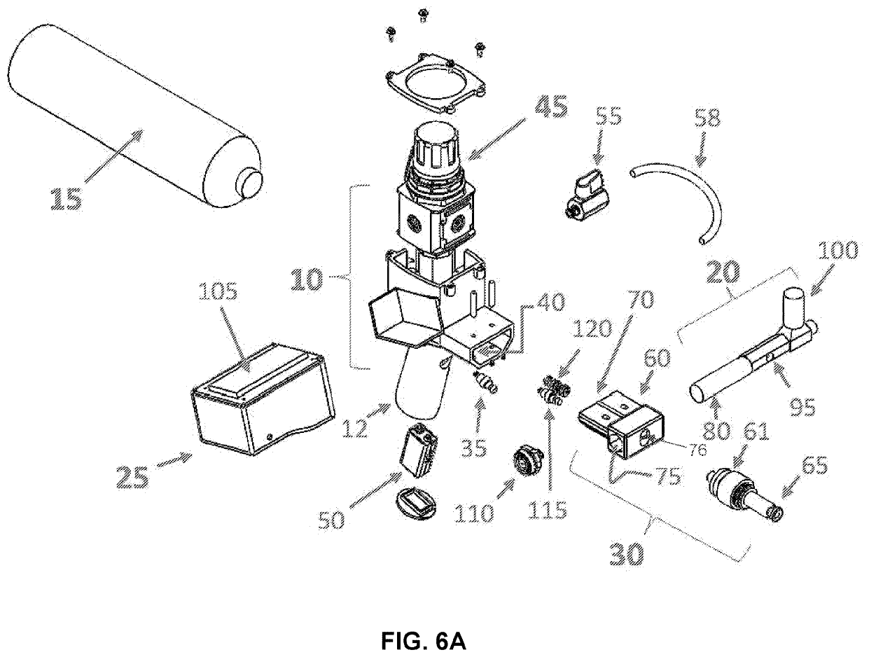

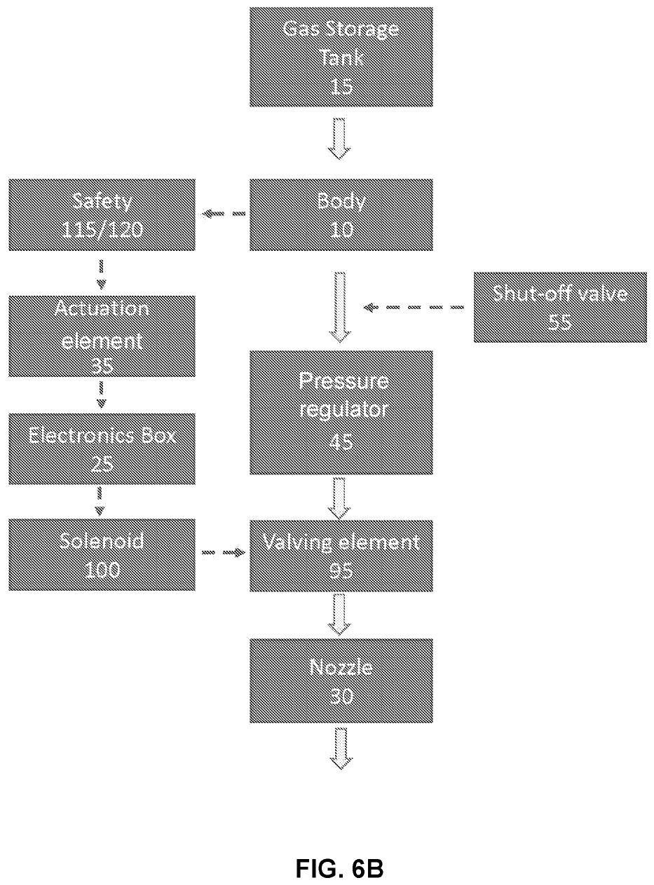

Referring to FIG. 6A, body 10 connects to all of the subsystems (e.g., gas storage tank 15, gas metering assembly 20, electronics box 25, nozzle 30, etc). The proximalmost end of gas storage tank 15 will be considered to be the most proximal point of device 5 when attached to body 10. At the opposite end of body 10 there comprises an internal chamber 40 for receiving the proximal end of nozzle 30, as will hereinafter be discussed in further detail. The part of nozzle 30 that comes in contact with the patient is therefore the most distal point of device 5. Body 10 comprises a handle 12 for a user to grip during use.

Body 10 further comprises an actuation element 35 for releasing a stream of compressed gas through nozzle 30, as will hereinafter be discussed in further detail. If desired, body 10 may further comprise a manual pressure regulator 45 (e.g., an adjustment dial) for permitting manual control over the gas pressure delivered through nozzle 30 when actuation element 35 is activated, as will hereinafter be discussed in further detail.

In one preferred form of the invention, handle 12 of body 10 further comprises a power source 50 (e.g., a 9V battery) for powering electronics box 25, as will hereinafter be discussed in further detail. Body 10 comprises a manual gas shut-off valve 55 that is connected by a hose 58 to gas metering assembly 20 which comprises a valving element 95 and a solenoid 100, for moving valving element 95 (e.g., a 3-way valve that shunts gas into meter chamber 80 and exhausts gas out of nozzle 30 as desired) to allow gas flow through nozzle 30, as will hereinafter be discussed in further detail. See FIGS. 2-5 and 6A.

### Electronic Components

Looking still at FIG. 6A, electronics box 25 preferably comprises a CPU and appropriate circuitry (not shown) for controlling solenoid 100 (whereby to permit the clinician to electronically control the release of pressure through nozzle 30), for reading pressure from a pressure sensor 110 indicative of the pressure inside a gas metering assembly 20 that is connected to valving element 95, and an LCD screen 105 for providing information to the operator (e.g., gas pressure to be delivered, gas remaining in gas storage tank 15, number of actuations of device 5, etc.). It can be appreciated that the actuation of device 5 can be done manually (i.e., without electronics) in another form of the invention by using single use disposable gas cylinders.

### The Gas Storage and Fluid Connections

Referring to FIGS. 6A and 6B, gas storage tank 15 is preferably a removable cylinder attached to body 10 via an appropriate means (e.g., a screw thread connection) so as to permit gas storage tank 15 to be replaced as needed. In one preferred form of the invention, gas storage tank 15 is mounted to body 10 so as to be fluidically connected to manual pressure regulator 45 which is, in turn, fluidically connected to the proximal end of nozzle 30. Shut-off valve 55 is preferably interposed in the path between gas storage tank 15 and nozzle 30 (e.g., to provide a safety for shutting off gas flow during storage of epidermal delivery device 5) as discussed above. It should be appreciated that, if desired, manual pressure regulator 45 may be replaced with a fixed pressure regulator, in which case, gas storage tank 15 is fluidically connected to nozzle 30 via a passage formed within body 10 (or via appropriate tubing disposed 58 therein). Gas storage tank 15 preferably comprises compressed helium gas (which is preferred due to its density), although other gasses may be used and will be apparent to those of ordinary skill in the art in view of the present disclosure. By way of example but not limitation, such other gasses may comprise ambient air, nitrogen gas, carbon dioxide and/or argon gas.

### The Nozzle

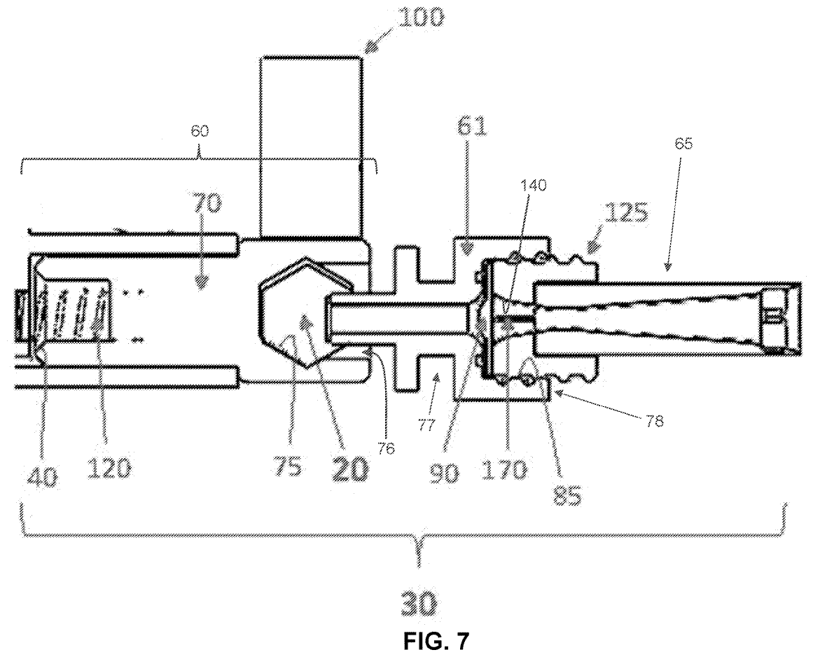

Referring to FIGS. 6A, 7 and 8, nozzle 30 comprises a proximally disposed body connection element 60 and a removable distal delivery tube 65. Body connection element 60 comprises a proximal end 70 shaped to be slidably received within internal chamber 40 of body 10, a gas metering assembly chamber 75 for receiving the gas metering assembly 20 and a nozzle chamber 76 for receiving a nozzle connection element 61. Nozzle connection element 61 is configured to permit selective mounting/dismounting of distal delivery tube 65 to body 10. More particularly, nozzle connection element 61 comprises a proximal end 77 for seating in nozzle chamber 76 of body connection element 60 and a distal end 78 comprising a chamber 85 for receiving removable distal delivery tube 65 via a delivery tube connector 125. Delivery tube connector 125 comprises a blister seat 87 for seating a blister 90 containing a powdered agent (e.g., a particle vaccine, a medicament, etc.), as will be discussed in further detail below.

### The Blade and Delivery Tube

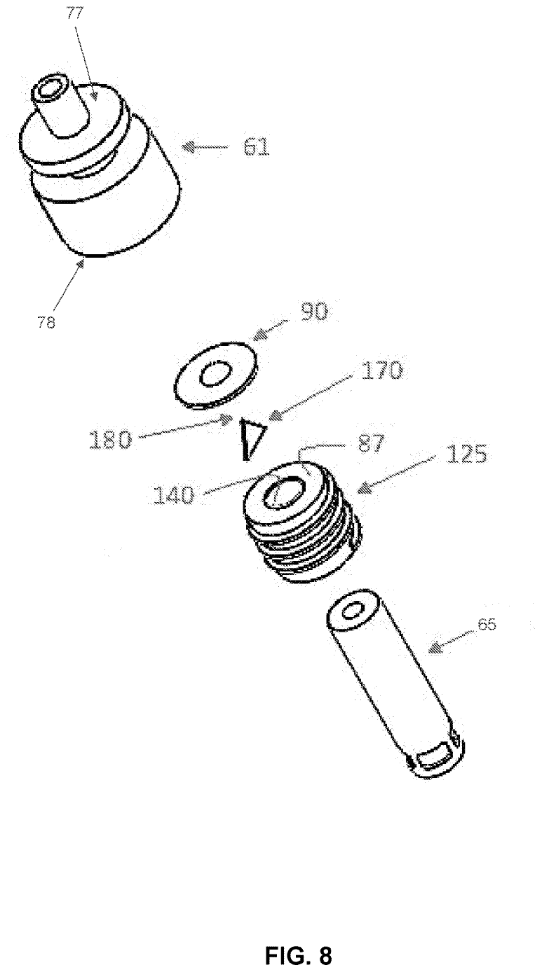

Looking now at FIG. 8, delivery tube connector 125 comprises an internal cavity 140 which conceals a blade element 170 and receives distal delivery tube 65. Preferably, seat 87 for receiving blister 90 is disposed at the proximal end of delivery tube connector 125 so that blister 90 is situated directly above blade element 170 and internal cavity 140.

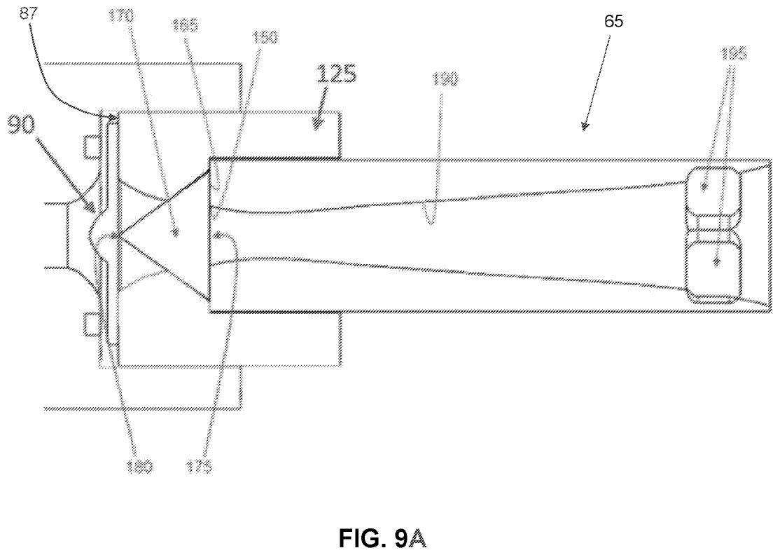

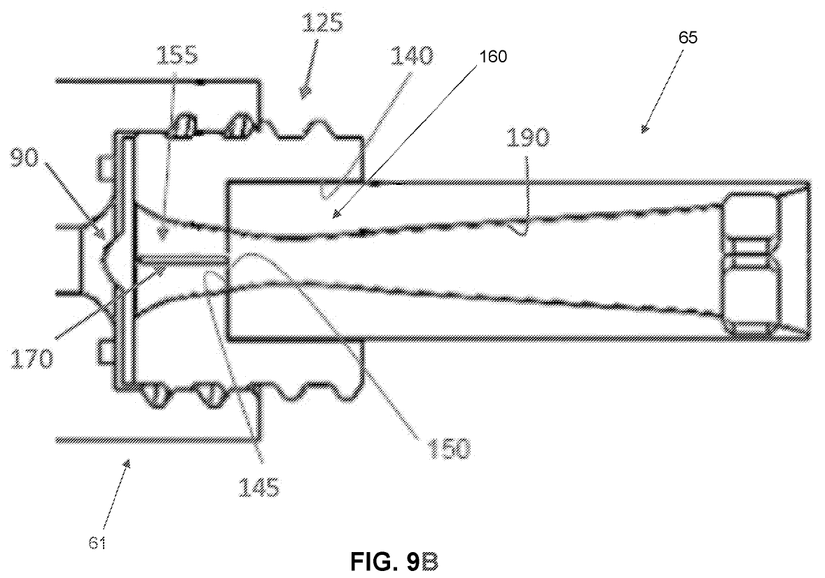

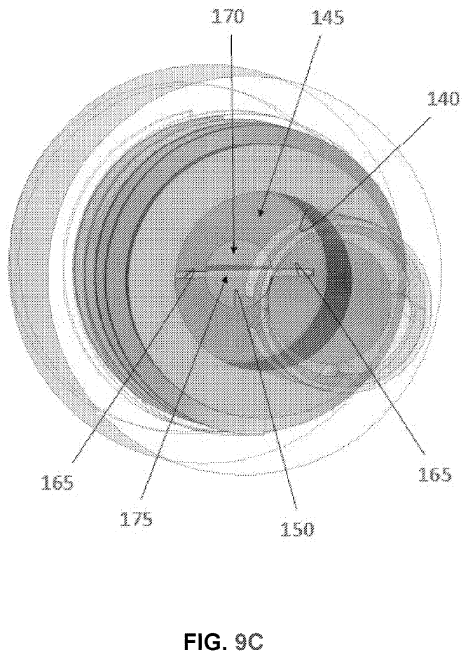

Referring now to FIG. 9A, blade element 170 generally comprises a flat distal end 175 and a pointed proximal tip 180 for piercing a blister 90, as will hereinafter be discussed in further detail. Rotating the cross-sectional view of distal delivery tube 65 and referring to FIG. 9B, a radially-extending shoulder 145 comprising a central opening 150 divides internal cavity 140 of delivery tube connector 125 into a proximal portion 155 and a distal portion 160. Radially-extending shoulder 145 comprises a pair of diametrically-opposed slots 165 sized to receive blade element 170, as depicted in FIG. 9C.

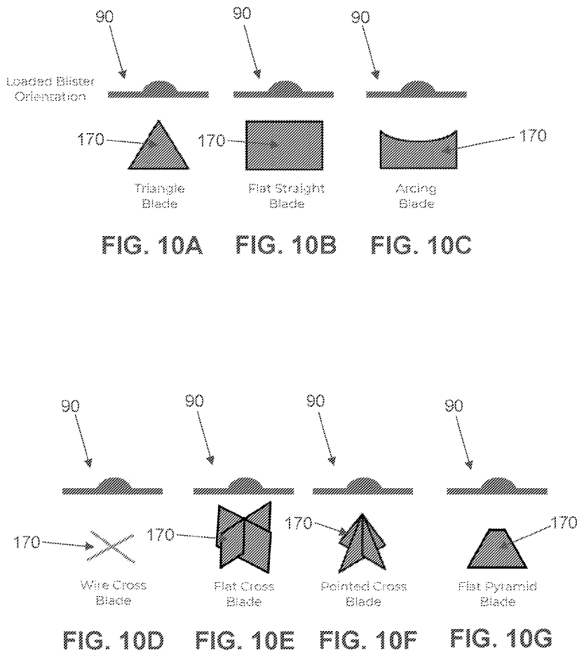

In a preferred embodiment of the invention, blade element 170 comprises a triangular cutting element (FIG. 10A). However, and looking now at FIGS. 10B-10G, if desired, blade element 170 may comprise other shapes/geometries such as a flat straight blade (FIG. 10B), an arcing blade (FIG. 10C), a wire cross blade (FIG. 10D), a flat cross blade (FIG. 10E), a pointed cross blade (FIG. 10F), or a flat pyramid blade (FIG. 10G). Other suitable shapes/geometries for blade element 170 will be apparent to one of skill in the art in view of the present disclosure.

### The Blister Packaging

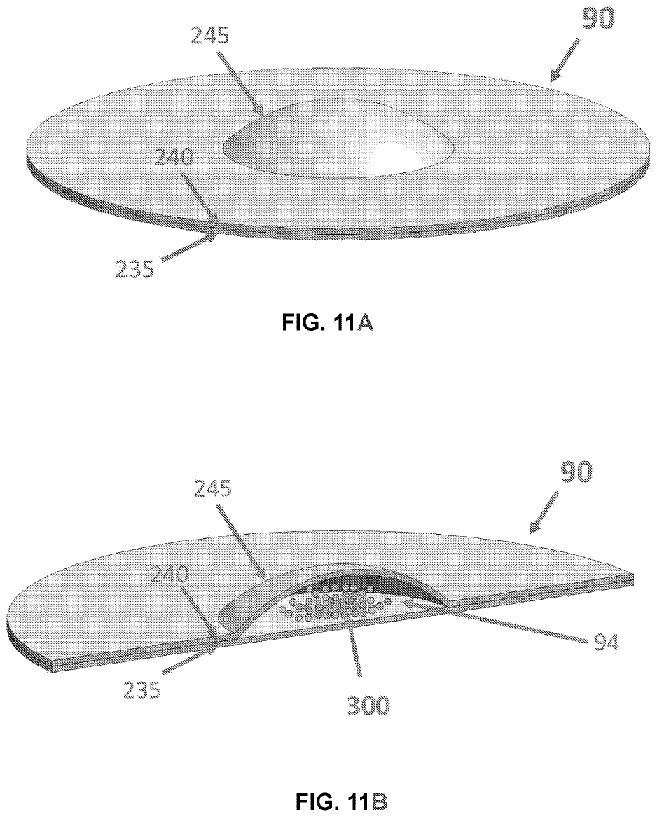

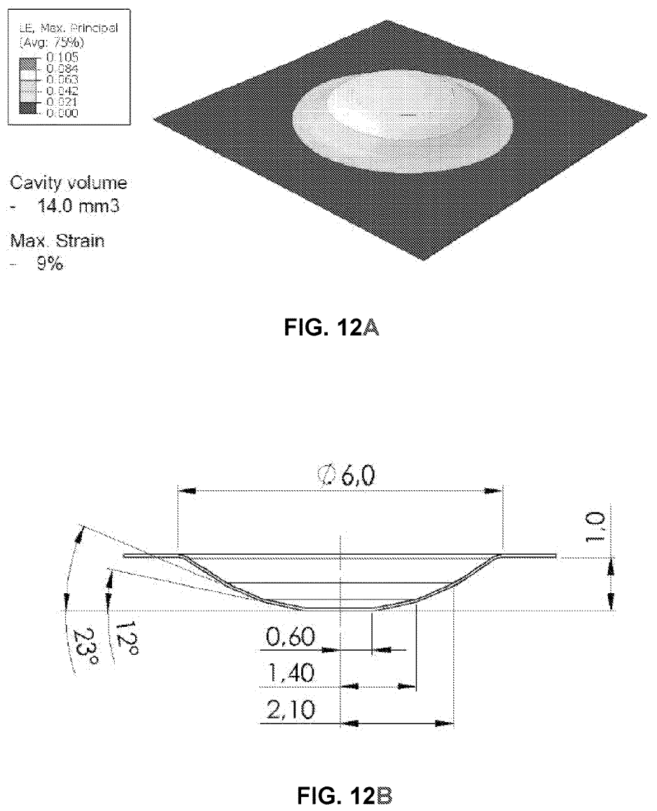

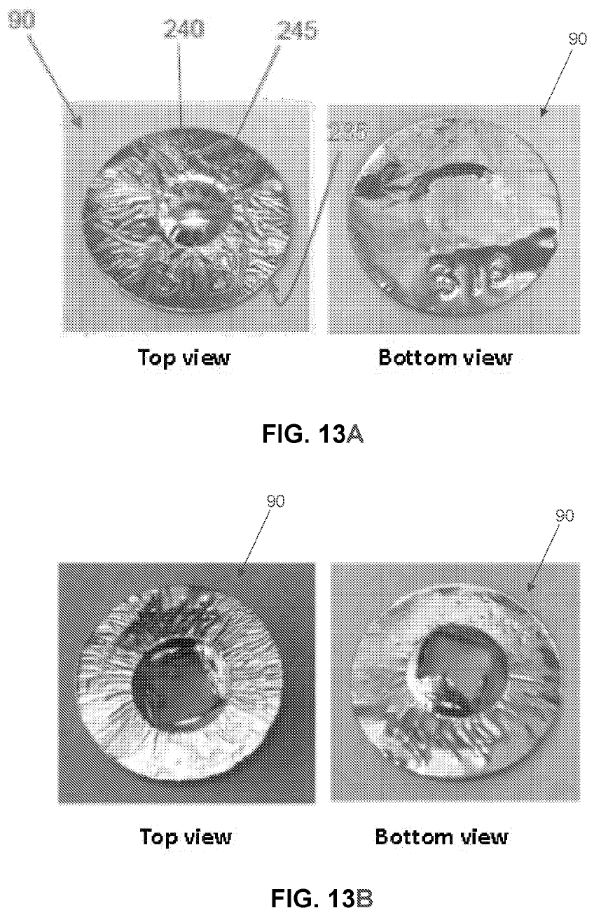

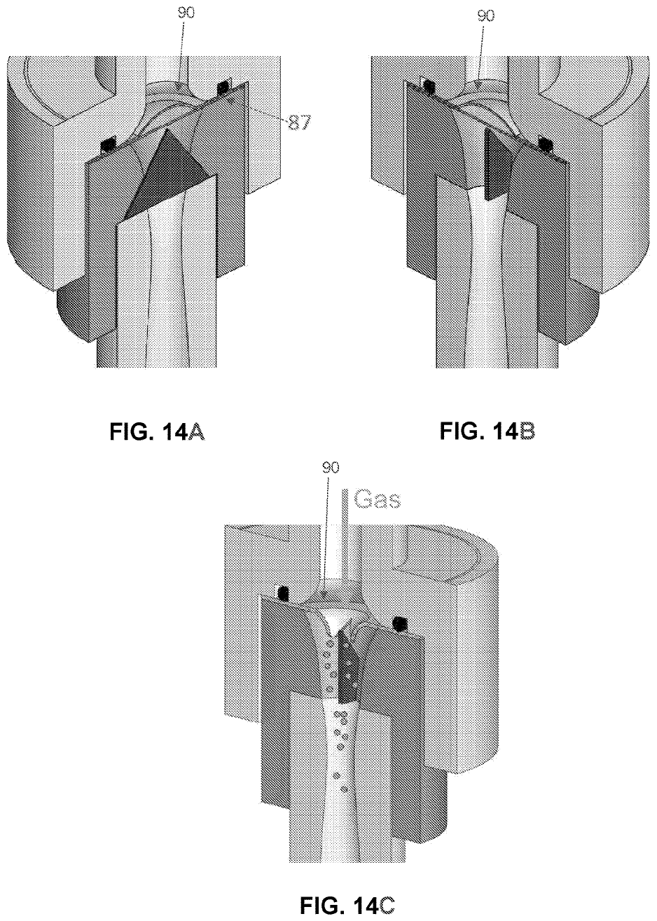

Looking now at FIGS. 11A and 11B, there is shown an exemplary particle vaccine packaging blister 90 formed in accordance with the present invention. Blister 90 is preferably circular in shape, and is preferably formed by press-sealing a bottom layer 235 of heavy-duty aluminum foil to a top layer 240 of heavy-duty aluminum foil with adhesive in a manner which creates a concave dome 245 forming a blister chamber 94 between bottom layer 235 and top layer 240 for receiving particle vaccine 300. Production of blisters 90 may be also ultrasonically welded, adhesive bonded, or heat sealed by some other means. Prior to sealing, particle vaccine 300 is weighed and filled in the blister chamber 94 of blister 90. Various designs and dimensions for blister 90 have been assessed and used in simulations to determine rupturing patterns and measure blister deformation, as will herein be discussed in further detail. FIGS. 12A and 12B show the shape, dimensions, and angles of blister 90 and concave dome 245 when formed for use with the present invention. Prior to use, blister 90 can be provided in the form of an intact blister 90 (FIG. 13A). After use, blister 90 takes the form of a lanced blister 90 (FIG. 13B). During delivery, it should be appreciated that concave dome 245 typically inverts due to gas flow and actuation, as will hereinafter be discussed in further detail. FIGS. 14A and 14B depict the intact form of blister 90 (FIG. 14A), and the lanced state of blister 90 (FIG. 14B) in the context of the nozzle 30.

Referring to FIGS. 8, 11A and 11B, bottom layer 235 of blister 90 sits atop (i.e., proximal to) seat 87. Blister 90 is thus situated with concave dome 245 directly in the path of the gas flow and centered above pointed proximal tip 180 of blade element 170. In a preferred embodiment of the invention, blister 90 and distal delivery tube 65 (including blade 170) are fully integrated so that blisters 90 do not need to be loaded manually by the user, as will hereinafter be discussed in further detail.

### Blister Rupturing and Particle Flow

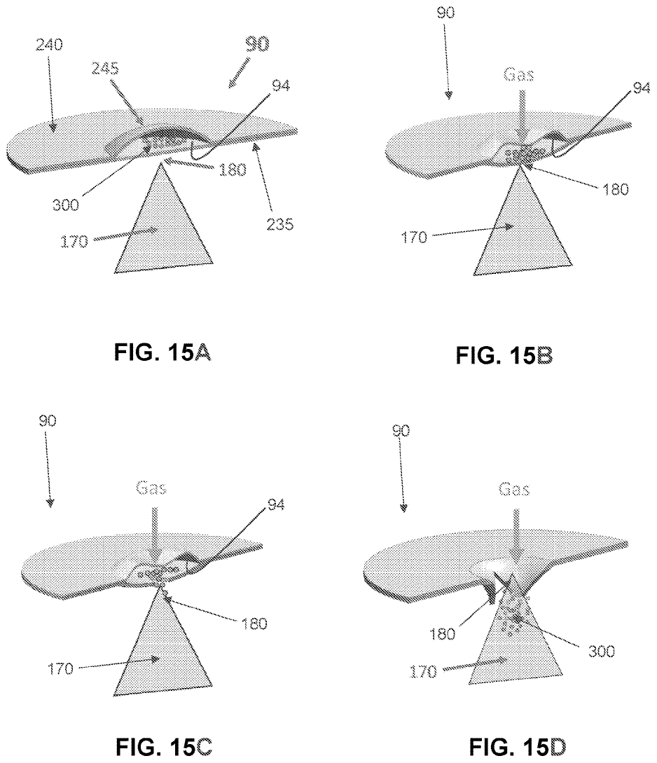

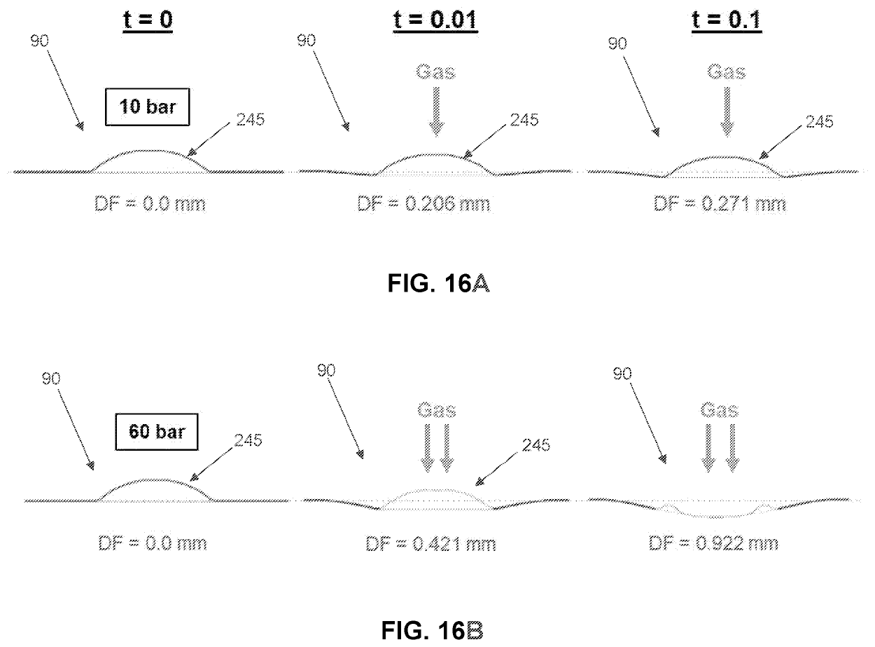

Looking now at FIGS. 15A-15D, when the present invention is actuated to permit the release of pressurized gas, gas will deform the intact blister so that the concave dome 245 deforms and inverts distally towards the tip of the blade 180 (FIG. 15B). Bottom layer 235 of blister 90 simultaneously moves towards pointed proximal tip 180 of blade element 170 and is pierced first (FIG. 15C), while top layer 240 and inverted dome 245 of blister 90 are pierced subsequently. The lancing of blister 90 gives access for particle vaccine 300 contained in blister chamber 94 to become entrained in the jet stream of gas and flow around blade 170 (FIG. 15D).

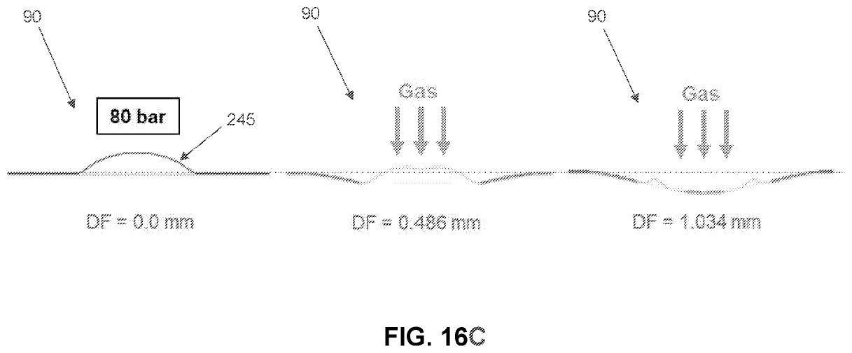

Simulations have been conducted to determine the effect of pressure on the deformation distance (DF) of blister 90 (see FIGS. 16A-16C). Shown are the DF at three timepoints (t=0, 0.01, and 0.1 sec) at three different pressures starting from 10 bar (FIG. 16A) to 60 bar (FIG. 16B) to 80 bar (FIG. 16C). At 10 bar, the pressure is not strong enough to invert concave dome 245 of blister 90 towards blade element 170.

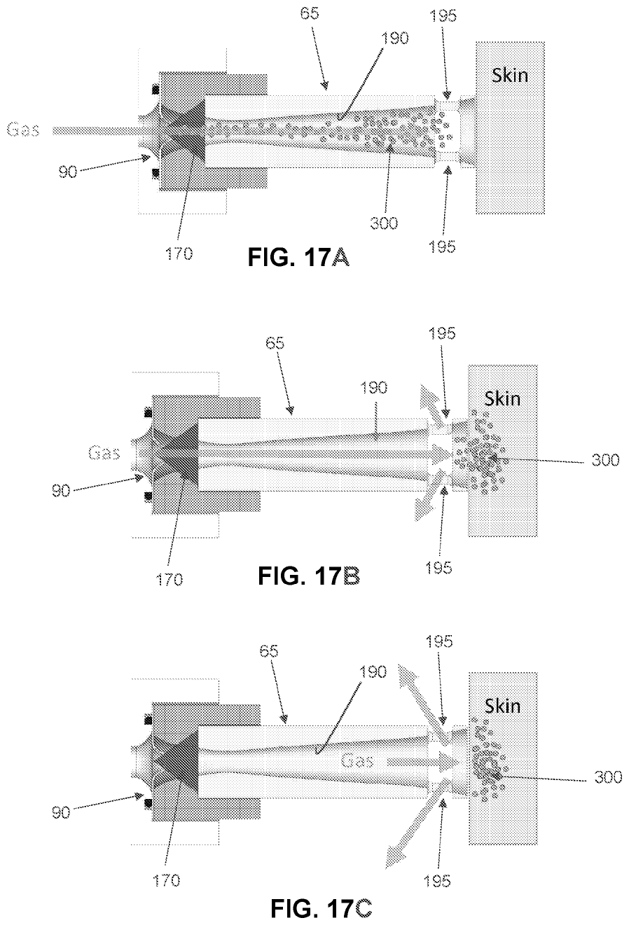

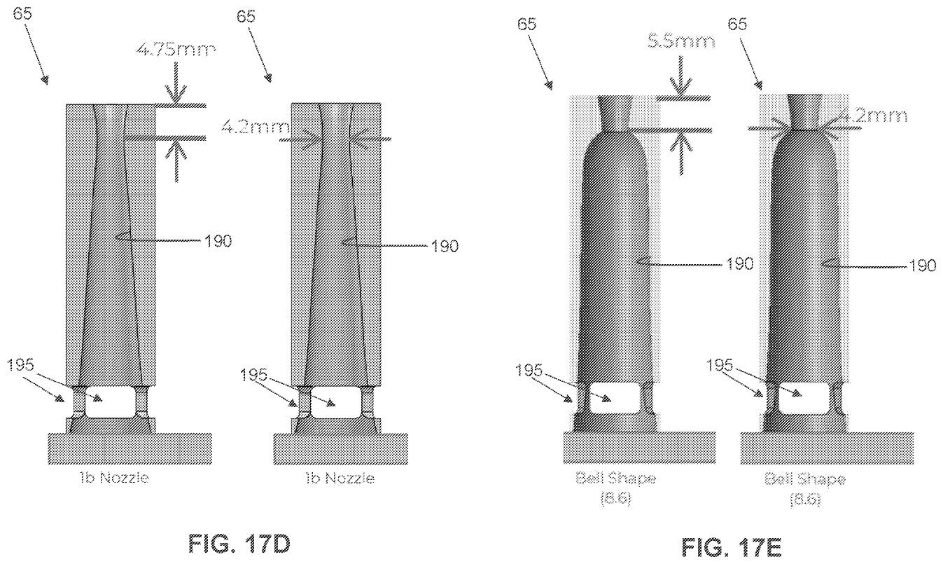

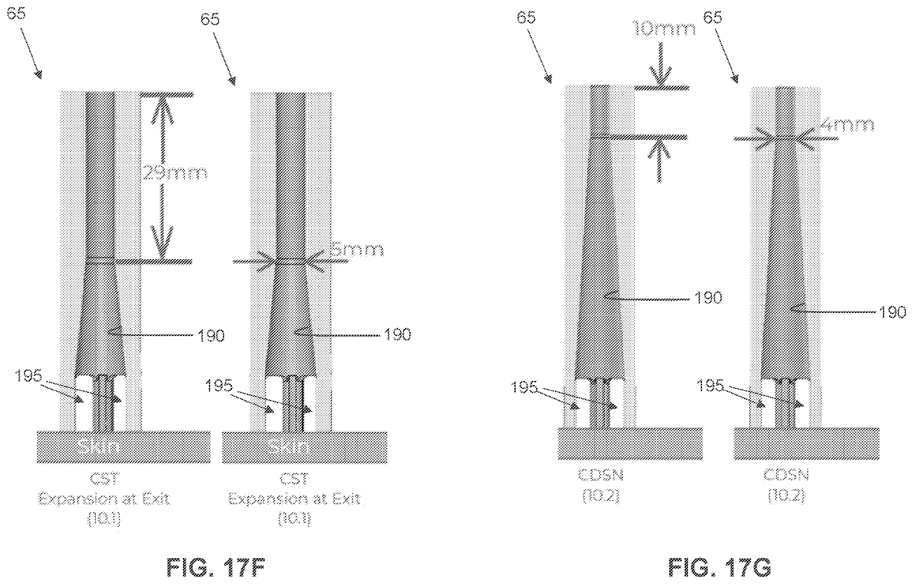

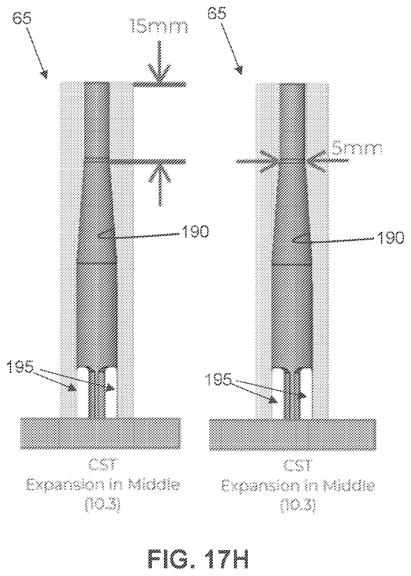

Referring to FIGS. 17A-17C, the particles of particle vaccine 300 that are entrained in the gas jet stream (FIG. 17A) move towards the skin with enough momentum to penetrate the skin barrier via the convergent and divergent internal lumen 190 of distal delivery tube 65 (FIG. 17B). In this embodiment of the invention, there are gas exhaust ports located at the distal end of internal lumen 190 of distal delivery tube 65 which allow gas to dissipate after actuation (FIG. 17C).

It will be appreciated that the foregoing construction achieves a high degree of entrainment of the particles of particle vaccine 300 within the gas jet stream such that less than 20% of particle vaccine 300 remains within blister 90 after blister 90 has been pierced in the manner discussed above. More particularly, it has been found that 85%±10% of particle vaccine 300 is successfully entrained in the gas jet stream when blister 90 is pierced in the manner discussed above.

### Internal Delivery Tube Flow Design

With the present invention, the powdered agent contained in blister 90 (e.g., particle vaccine 300) is entrained in the jet stream and are propelled to a specific momentum to permit skin penetration and the delivery of the vaccine within a certain volume of the epidermis (FIG. 1).

Referring back to FIG. 9B, the proximal end of distal delivery tube 65 is mounted within internal cavity 140 of delivery tube connector 125 so as to extend distally therefrom. Distal delivery tube 65 comprises an internal lumen 190 radially aligned with central opening 150 of radially-extending shoulder 145. In a preferred form of the invention, internal lumen 190 comprises a variable diameter along the longitudinal axis of distal delivery tube 65. By way of example but not limitation, internal lumen 190 may comprise a smaller diameter at the proximal end of distal delivery tube 65 (i.e., where internal lumen 190 is aligned with central opening 150 of radially-extending shoulder 145), and a larger diameter at the distal end of distal delivery tube 65 (i.e., where distal delivery tube 65 is pressed against the skin of person to be injected with the contents of blister 90, as will hereinafter be discussed in further detail). Furthermore, if desired, internal lumen 190 may comprise one or more internal constrictions along its longitudinal axis, such that the diameter of internal lumen 190 is flared at one or both of the distal and proximal ends of distal delivery tube 65. The convergent and divergent internal lumen designs of distal delivery tube 65 facilitates the acceleration of the particles through the isentropic flow effect.

In a preferred form of the invention, a plurality of ports 195 (see FIGS. 17A-17C) are formed in the sidewall of the distal end of distal delivery tube 65 and in fluid communication with internal lumen 190, whereby to permit the escape of excess gasses in the area of contact with the patient's skin and enhance the stability of nozzle 30 relative to the patient's skin, as will hereinafter be discussed in further detail.

FIGS. 17D-17H show various exemplary constructions for distal delivery tube 65 formed in accordance with the present invention. FIGS. 17D-17H show exemplary locations for internal constrictions along the longitudinal axis of internal lumen 190, i.e., depicted in FIGS. 17D-17H as distances from the proximal end of each delivery tube 65. FIGS. 17D-17H also show exemplary preferred dimensions for internal constrictions along the longitudinal axis of internal lumen 190, i.e., shown as distances from the outermost wall of each delivery tube 65 (which wall is parallel to the longitudinal axis of internal lumen 190) toward the internal constriction. FIGS. 17D-17H also disclose exemplary arrangements for ports 195 formed in the sidewall of the distal end of distal delivery tube 65. The construction of FIG. 17F represents the preferred construction for distal delivery tube 65.

### Safety Mechanism

In one preferred form of the invention, and referring back to FIGS. 6A and 6B, electronics box 25 is electrically connected to a safety mechanism disposed within internal chamber 40 and is configured to permit solenoid 100 to open valving element 95 (and thereby permit gas flow through distal delivery tube 65) when the safety mechanism is disengaged, so as to prevent accidental discharge of gas through nozzle 30.

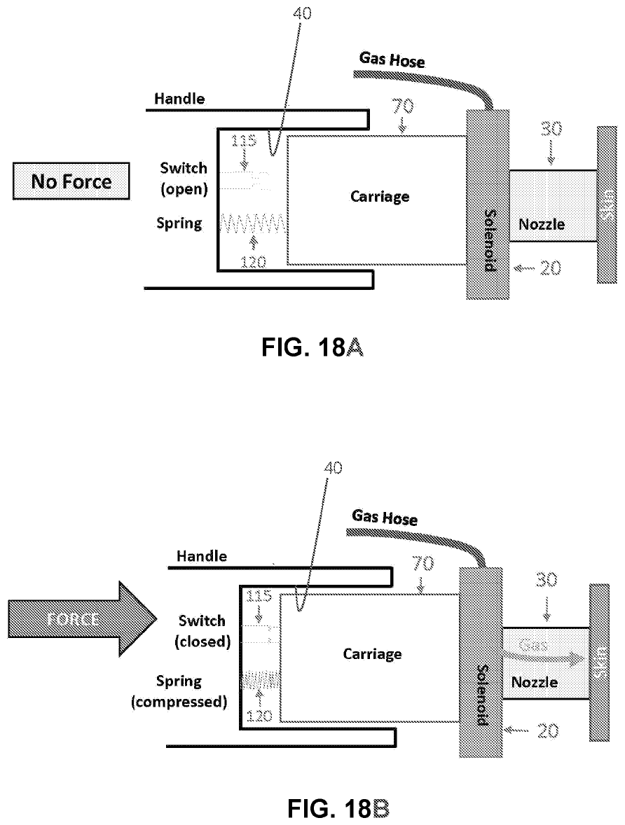

Now looking at FIGS. 6A, 18A and 18B, if desired, internal chamber 40 may comprise a sensor switch 115 disposed at the proximal end of internal chamber 40 for detecting when nozzle 30 is slidably disposed within internal chamber 40 at its proximal-most position. In this form of the invention, one or more springs 120 are preferably interposed between the proximal-most end of nozzle 30 and the distal wall of internal chamber 40 so as to bias nozzle 30 distally, as shown in FIG. 18A when no force is applied. Only when force has been applied to nozzle 30, as shown in FIG. 18B, and the distal wall of internal chamber 40 has been pushed proximally against the power of spring(s) 120 so as to contact sensor 115, can the present invention be used.

In use, when the distal delivery tube 65 is disposed against the skin of the patient, the clinician pushes handle 12 distally such that proximal end 70 of body connection element 60 slides proximally within body 10, whereby to engage sensor 115, thereby completing the electronic circuit of the safety mechanism such that solenoid 100 is permitted to open valving element 95, whereby to permit gas to flow to distal delivery tube 65.

### Alternative Distal Delivery Tube Mounting Designs

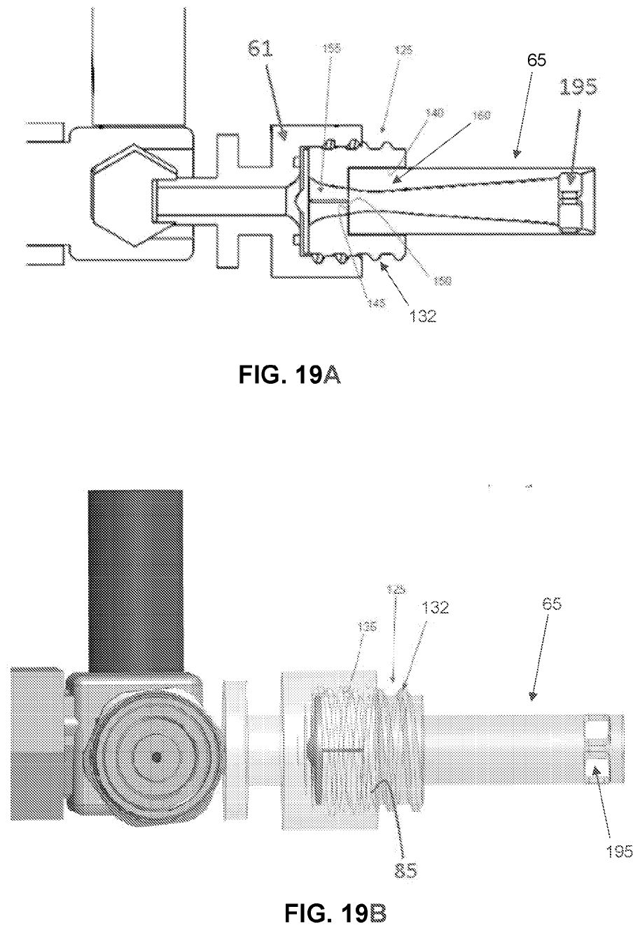

Looking now at FIGS. 19A and 19B, there are shown alternative ways of connecting the distal delivery tube 65 to body 10 of the present invention. More particularly, distal delivery tube 65 comprises a delivery tube connector 125 for releasably mounting distal delivery tube 65 to nozzle connection element 61. In one form of the invention (FIGS. 19A and 19B), delivery tube connector 125 comprises a thread 132 for threadingly engaging a counterpart thread 135 formed in the sidewall of chamber 85 of nozzle connection element 61. However, it should be appreciated that, if desired, alternative designs may be used in place of a threaded connection to mount the distal delivery tube 65 in a manner that will be appreciated by one of ordinary skill in the art in view of the present disclosure.

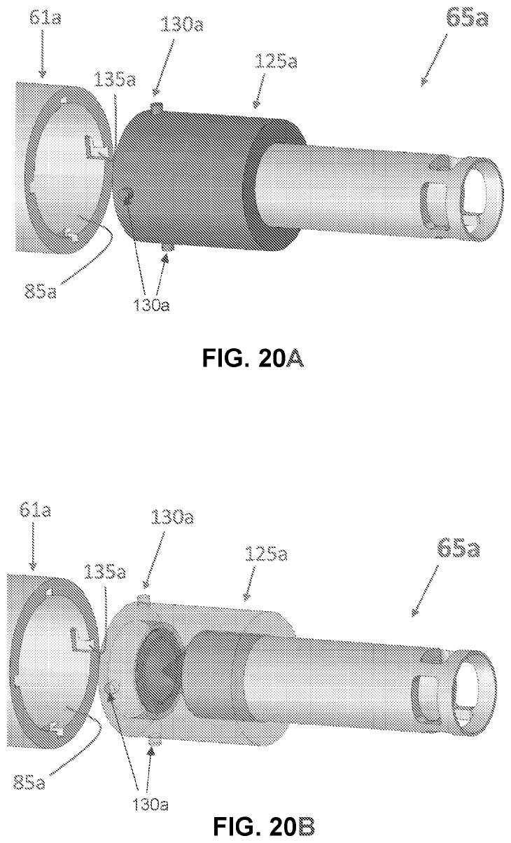

By way of example but not limitation, and looking now at FIGS. 20A and 20B, if desired, a twist mount (e.g., a “bayonet” mount) design may be used to mount an alternative distal delivery tube 65a via a delivery tube connector 125a and nozzle connection element 61a. One or more extrusions 130a on delivery tube connector 125a are configured to slide fully into one or more insets 135 a (e.g., L-shaped slots) found within chamber 85 of nozzle connection element 61a and to then be rotated into a locking position. To dismount a twist mount distal delivery tube 65a, the user rotates (i.e., “twists”) distal delivery tube 65a in the opposite direction and disconnects the disposable tube from body 10 by sliding extrusions 130a out of insets 135a while moving distal delivery tube 65a distally.

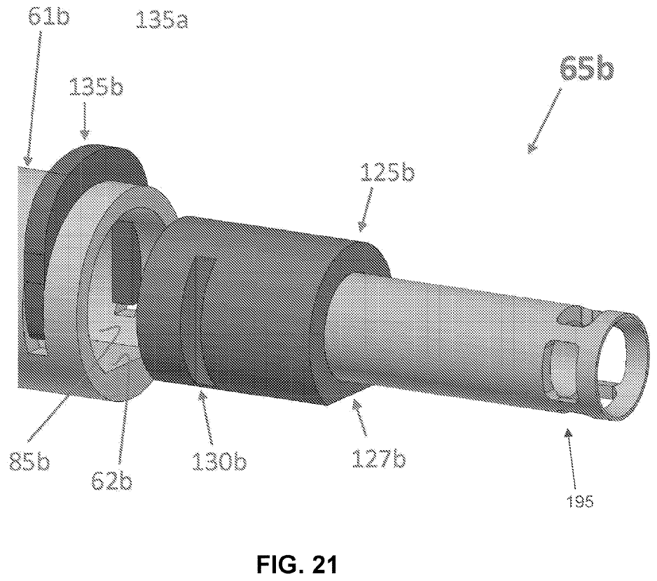

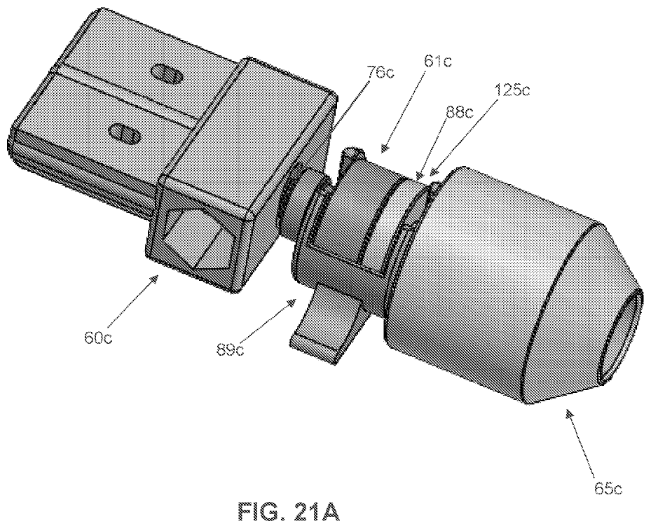

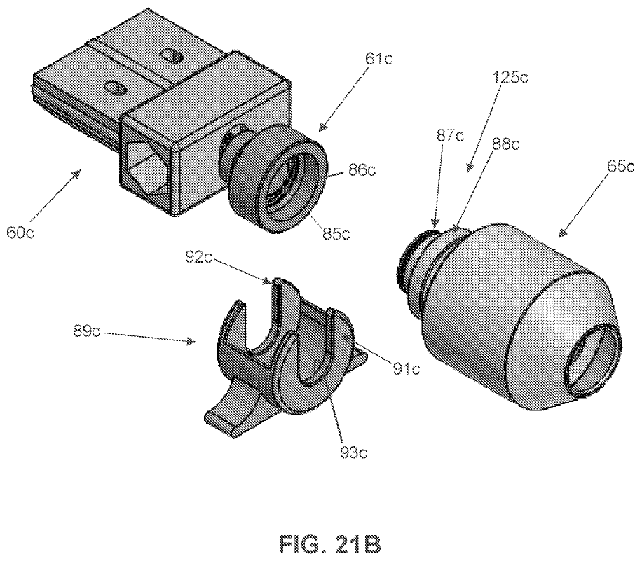

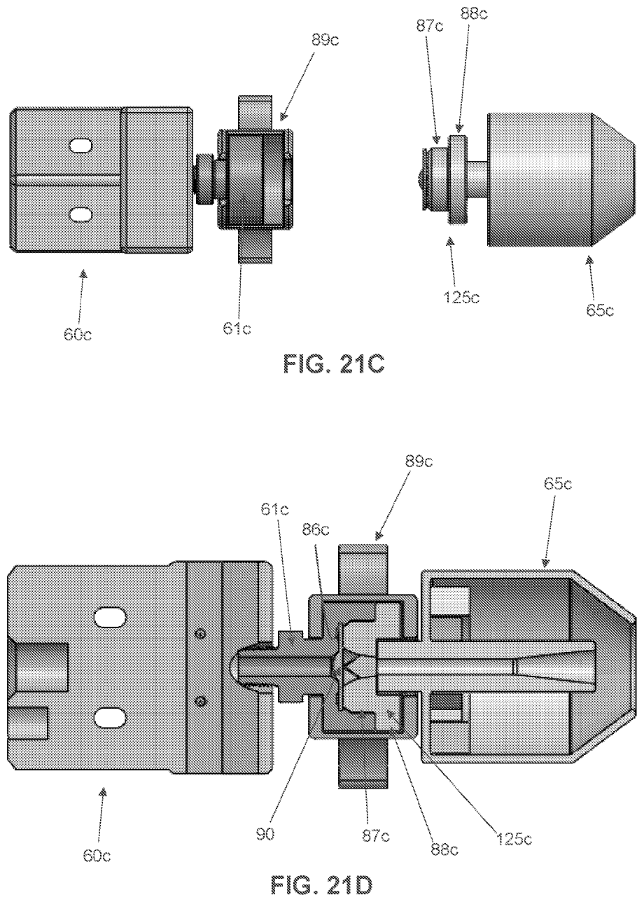

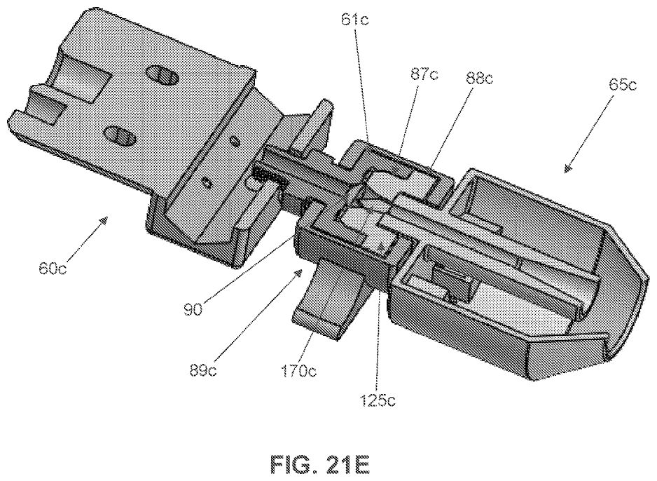

By way of further example but not limitation, and looking now at FIG. 21, if desired, a “snap mount design” may be used to mount an alternative distal delivery tube 65b via a delivery tube connector 125b and a nozzle connection element 61b. To mount distal delivery tube 65b, delivery tube connector 125b must slide into chamber 85b of nozzle connection element 61b by (i) ensuring that the flat 127b of delivery tube connector 125b aligns with counterpart flat 62b of chamber 85b of nozzle connection element 61b, and (ii), applying enough force to snap distal nozzle connection clip 135b within the proximal connector inset 130b of delivery tube connector 125b. To dismount the snap mount tube 65b, the distal nozzle connection clip 135b must be moved back to the starting position so that the delivery tube connector 125b can be disconnected from nozzle connection element 61b of body 10.

By way of still further example but not limitation, and looking now at FIGS. 21A-21E, there is shown another novel distal delivery tube 65c formed in accordance with the present invention. Distal delivery tube 65c comprises a delivery tube connector 125c for disposition in chamber 85c of nozzle connection element 61c. Chamber 85c comprises a circumferentially-extending groove 86c (FIG. 21B) defined by radially-tapered sidewalls and sized to receive the outer edge of blister 90 seated on seat 87 of delivery tube connector 125c, whereby to permit blister 90 to be “snapped” into groove 86c and retained therein, even when chamber 85c is upended (i.e., relative to Earth gravity). A proximal flange 87c extends proximally from the proximal end of delivery tube connector 125c, and is sized to be slidably received within chamber 85c and to make a sealing fit with the side wall thereof (FIG. 21E). A distal flange 88c is disposed distal to proximal flange 87c and extends radially outboard thereof. A snap clamp 89c comprising an upper flange 91c, a lower flange 92c, and a cutout 93c formed in both of flanges 91c, 92c. Snap clamp 89c is configured such that upper flange 91c sits distal to distal flange 88c when proximal flange 87c is moved into chamber 85c, and such that lower flange 92c sits proximal to the proximalmost radially-outboard-extending surface of nozzle connection element 61c, whereby to releasably mount delivery tube connector 125c within chamber 85c of nozzle connection element 61c. When it is desired to dismount delivery tube connector 125c from chamber 85c, snap clamp 89c is removed, and proximal flange 87c is moved out of chamber 85c so as to allow access to blister 90. Blister 90 may thereafter be removed from groove 86c and replaced with a new (i.e., un-pierced) blister 90.

### Alternative Delivery Tube Designs

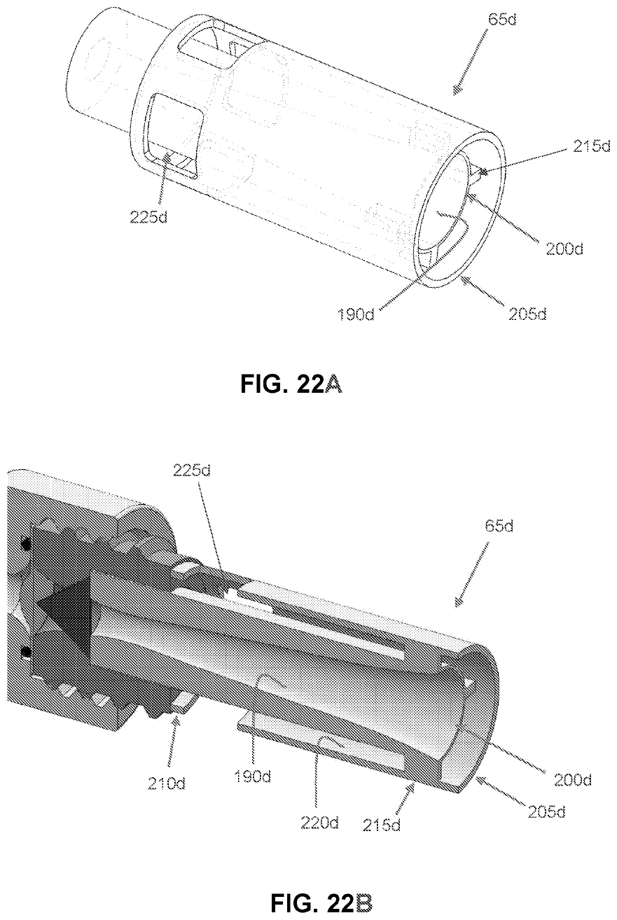

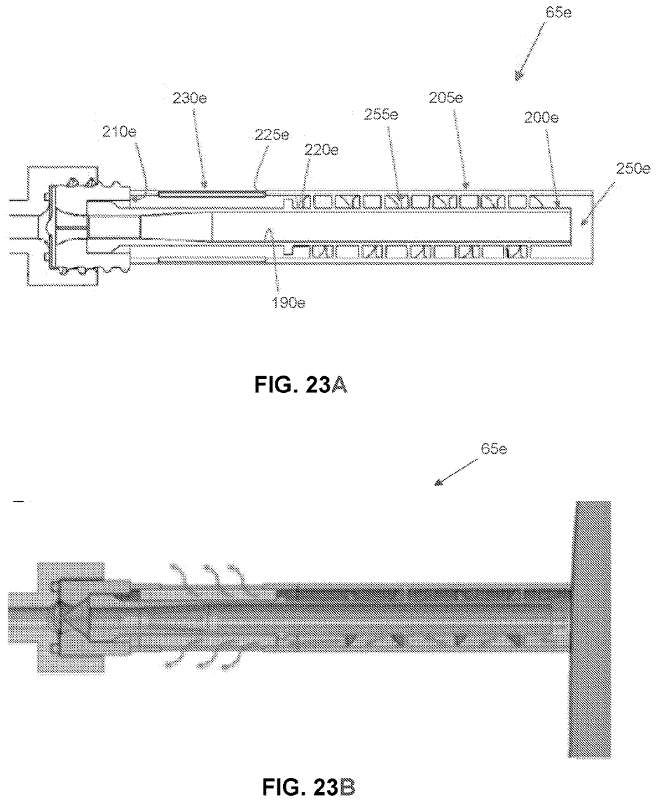

Looking now at FIGS. 22A, 22B, 23A, 23B, 24A and 24B, there are shown still further alternative designs for distal delivery tube 65. For example, FIGS. 22A and 22B show a double-walled distal delivery tube 65d that is designed to prevent the escape of particle vaccine into the environment. In this form of the invention, lumen 190d of distal delivery tube 65d is defined by an inner wall 200d and an outer wall 205d extending along all of, or a portion of, inner wall 200d. A circumferentially-extending proximal end cap 210d connects outer wall 205d to inner wall 200d at the proximal end, while a plurality of circumferentially-extending segments 215d (e.g., four segments 215d) connect outer wall 205d to inner wall 200d at the distal end. Outer wall 205d, inner wall 200d, proximal end cap 210d and circumferentially-extending segments 215d together define a circumferentially-extending chamber 220d disposed therebetween. One or more ports 225d are preferably formed in a proximal area of outer wall 205d in fluid communication with chamber 220d. In one preferred form of the invention a filter (not shown) can be disposed over, or within, each port 225d so as to capture any medicament ejected out of each port 225d, as will hereinafter be discussed in further detail.

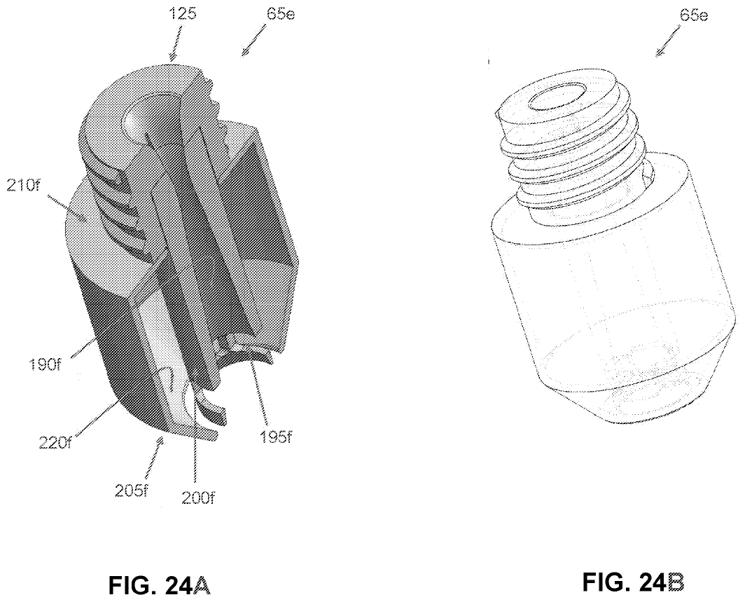

Looking now at FIGS. 23A and 23B, there is shown an alternative nozzle design comprising a double-walled, elongated distal delivery tube 65e that comprises multiple baffles 255e throughout the length of distal delivery tube 65e. When the device is actuated, a burst of metered gas is emitted and flows distally through the lumen 190e of distal delivery tube 65e towards the skin. After reaching the skin, the gas will bounce and flow to the area of least resistance (i.e., chamber 220e) and then will flow proximally through chamber 220e created by the outer wall 205e. The channels and baffles 255e cause the gas flow to be disrupted and convoluted, thus slowing down the velocity of gas so that the gas exits through ports 225e at reduced exit speed, and therefore with reduced noise. Furthermore, as a result of this construction, any residual powder/particles (e.g., the medicament still suspended in the jet of gas that is not delivered through the skin of the patient) is passed back through chamber 220e (i.e., away from the patient) and through one or more filters 230e before being exhausted to the ambient air. This construction both moves the jet of gas exhaust away from the patient and ensures that the powder/particles are not dispersed into ambient air where it could cause a deleterious effect (e.g., be inhaled by the patient or the clinician).

As shown in FIGS. 24A and 24B, in another form of the present invention, outer wall nozzle exhaust ports 195f are located at the distal end of distal delivery tube 65f (as it is in distal delivery tube 65). A double-walled version of distal delivery tube 65f comprises an inner wall 200f surrounded circumferentially by an outer wall 205f that is connected by a circumferentially extending proximal shoulder cap 210f. See FIG. 24A. The larger area in this design concept would allow for gas expansion to occur and reduce the noise emitted from the device. Furthermore, any excess particle vaccine that is entrained in the gas flow through the lumen 190f but which does not penetrate the skin, will exit through exhaust ports 195f and be contained within chamber 220f to prevent dispersion.

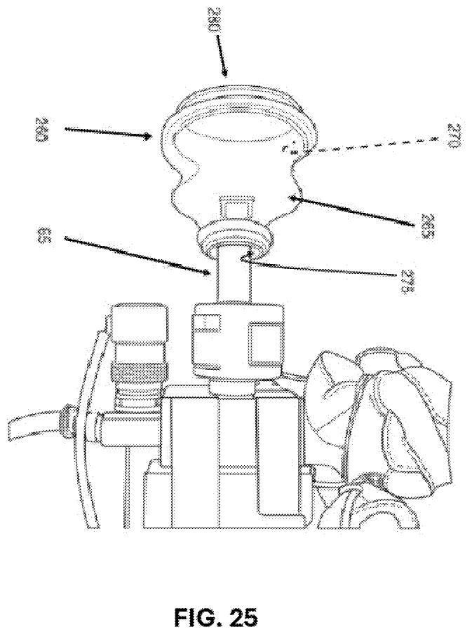

Looking now at FIG. 25, in another form of the present invention, there is provided a novel suction cup distal interface 260 formed in accordance with the present invention. Suction cup distal interface 260 generally comprises a resilient flexible body 265 defining a central cavity 270, a proximal opening 275 configured to permit distal delivery tube 65 to pass therethrough, and a distal opening 280 for pressing against the skin of the patient and making an airtight seal therewith, as will hereinafter be discussed in further detail.

More particularly, suction cup distal interface 260 is mounted over distal delivery tube 65 so that distal delivery tube 65 extends through proximal opening 275, with the body of distal delivery tube 65 residing within central cavity 270 being spaced from the sidewalls thereof, and with the distal end of distal delivery tube 65 being in the same plane as, or proximal to, the plane defined by the distalmost surface of suction cup distal interface 260 extending perpendicular to the longitudinal axis thereof.

In use, the clinician pushes distal delivery tube 65 and suction cup distal interface 260 against the skin of the patient so as to create a seal therewith. If desired, the clinician may “pinch” the resilient sidewall of suction cup distal interface 260 so as to effect a lower pressure inside cavity 270 then the ambient air pressure, whereby to facilitate sealing suction cup distal interface 260 to the skin of the patient.

### The Use of Novel Transdermal Delivery Device

In use, a clinician desiring to use the novel handheld transdermal delivery device 5 to deliver a powdered medicament (e.g., particle vaccine 300) to a patient begins by loading a blister 90 comprising the appropriate powdered medicament onto seat 87 of delivery tube connector 125 such that blister 90 is disposed with the bottom layer 235, facing distally towards the open end of central opening 150.

Distal delivery tube 65 is mounted to delivery nozzle connection element 61 via delivery tube connector 125. In the situation in which delivery tube connector 125 is already mounted inside chamber 85 of nozzle connection element 61, the clinician must first remove delivery tube connector 125 in order to gain access to blister seat 87 of delivery tube connector 125 so as to install a blister 90. Delivery tube connector 125 must thereafter be re-installed into chamber 85 of nozzle connection element 61 in order to use transdermal delivery device 5. Note that after use, the clinician can remove (or replace) blister 90 by dismounting delivery tube connector 125 from chamber 85 and removing the blister from seat 87. If desired, a new, unused blister can then be installed in seat 87 before delivery tube connector 125 is re-mounted within chamber 85 of nozzle connection element 61. As a result of this construction, the clinician is able to easily and quickly install new (and remove spent) doses of medicament, allowing novel transdermal delivery device 5 to be used in a highly repeatable manner.

Once blister 90 has been installed on seat 87 and delivery tube connector 125 has been mounted within chamber 85 of nozzle connection element 61, transdermal delivery device 5 may be used to deliver the powdered medicament transdermally to the patient as follows.

The clinician opens shut-off valve 55 and manual pressure regulator 45 (if provided) in order to establish the flow of gas from gas storage tank 15 to gas metering assembly 20. The clinician then presses the distal end of distal delivery tube 65 against the skin of the patient at the site where the transdermal delivery of the medicament is to be effected. Where transdermal delivery device 5 comprises a safety mechanism, the clinician pushes handle 12 distally so that it bears against nozzle 30 and the skin of the patient, causing the proximal end of nozzle 30 to slide proximally within internal chamber 40 (or, causing internal chamber 40 to advance distally over the proximal end of nozzle 30) against the power of spring(s) 120 until sensor 115 is engaged by nozzle 30.

The clinician can then actuate actuation element 35 (e.g., where actuation element 35 comprises a trigger, the clinician pulls the trigger). Actuation of actuation element 35 opens the on-off solenoid 100 to fill the meter chamber 80 to the pressure set by pressure regulator 45 which is then is exhausted distally through valving element 95 and nozzle 30. The jet of gas then passes distally through nozzle connection element 61 to chamber 85, where the jet of gas encounters concave dome 245 of blister 90.

Inasmuch as blister 90 substantially blocks further distal passage of the emitted jet of gas, and inasmuch as blister 90 is made out of flexible foil, the force of the emitted jet of gas against the blister 90 (i) causes blister 90 to flex distally towards the tip of blade element 170, and (ii) causes concave dome 245 of blister 90 to invert distally. As these blister events occur, the bottom layer 235 is lanced by pointed proximal tip 180 and the inverted concave dome 245 of top layer 240 of blister 90 is lanced subsequently. It should be appreciated that as this occurs, the particle vaccine contained within the concave dome 245 is exposed to the flowing gas jet, which can now pass distally through the pierced blister 90, entraining the powdered medicament (e.g., particle vaccine 300) within the gas jet and accelerating the powdered medicament (e.g., particle vaccine 300) distally through internal lumen 190 of distal delivery tube 65.

When the powdered medicament entrained within the gas jet encounters the patient's skin at the open distal end of distal delivery tube 65, the powdered medicament has been accelerated to an appropriate velocity relative to the mass of the particular powdered medicament such that the particles of the powdered medicament have sufficient momentum to pass through the patient's skin and penetrate to the desired depth to be clinically effective. Excess gas and excess powdered medicament then passes out of one or more ports 195 formed in the distal end of distal delivery tube 65.

In the situation in which distal delivery tube 65 comprises a “double tube” construction (FIGS. 22A, 22B, 23A, 23B, 24A and 24B), excess gas and medicament is directed out ports 195, proximally back through chamber 220, and out one or more ports 225. As discussed above, one or more filters 230 may be disposed within ports 225 in order to capture excess medicament contained within the gas exhausted out of ports 225.

It will be appreciated that the foregoing method is substantially similar regardless of which form of distal delivery tube 65 (e.g., tube 65a, tube 65b, tube 65c, etc.) is utilized, and/or if the aforementioned suction cup distal interface 260 is utilized.

### An Exemplary, Compact Embodiment of the Invention

In an exemplary, compact embodiment of the present invention, epidermal delivery device 5 comprises a more portable version of the invention in a smaller footprint. In this form of the invention, gas storage tank 15 will not be removable and blister 90 containing particle vaccine 300 (and/or other therapeutics) is fully integrated into a sterile, disposable nozzle 30 so that the clinician does not need to replace blisters 90 between subsequent deliveries of particle vaccine 300 to patients, and instead replaces the entire nozzle 30. This ensures that there is no carry over of powdered medicament that may still reside within the lumen 190 of the nozzle 30 with subsequent use of the invention. The integrated blister and nozzle will be filled and packaged and contain the necessary medicament information (e.g., lot #, expiry date, dosage).

### Alternative Uses of the Novel Transdermal Delivery Device

While the foregoing description of the novel transdermal delivery device discusses using delivery device 5 in the context of needle-free delivery of vaccines through the skin of a patient, it should be understood that the novel transdermal delivery device of the present invention may be used to effect the needle-free delivery of substantially any other agents (e.g., including, but not limited to medicaments or pharmaceuticals) through the skin of a patient. By way of example, delivery device 5 may be used to perform needle-free cosmetic procedures that benefit from needle-free delivery of materials through the skin of a patient (e.g., needle-free delivery of BOTOX®).

### Modifications

It should be understood that many additional changes in the details, materials, steps and arrangements of parts, which have been herein described and illustrated in order to explain the nature of the present invention, may be made by those skilled in the art while still remaining within the principles and scope of the invention.

1. An apparatus for transdermal delivery of a powdered agent to a patient, the apparatus comprising:
a fluid source comprising a fluid;
a nozzle extending distally from the fluid source, the nozzle comprising a proximal end, a distal end and a lumen extending from the proximal end to the distal end;
a seat disposed at the proximal end of the nozzle;
a sealed blister with a concave dome proximal surface and a flat distal surface containing the powdered agent positioned on the seat of the nozzle disposed within the lumen;
a blade disposed distal to the seat with the lumen;
an actuation element for releasing the fluid from the fluid source, wherein the actuation element causes the released fluid to push the sealed blister forward, deforming the concave dome of the sealed blister to move distally toward the blade, and the blade pierces the sealed blister to release the powdered agent into the stream of fluid for entrainment and delivery through the distal end of the nozzle.

2. The apparatus according to claim 1 wherein the apparatus further comprises a pressure regulator for manually adjusting the pressure of the fluid.

3. The apparatus according to claim 1 wherein the fluid source comprises a single container configured to supply a plurality of fluid releases.

4. The apparatus according to claim 1, wherein the sealed blister comprises aluminum foil sealed to form a sealed blister chamber.

5. The apparatus according to claim 1 wherein the nozzle comprises a nozzle connector for mounting the nozzle to the fluid source and disposable delivery tube.

6. The apparatus according to claim 5 wherein the nozzle connector comprises a first connector portion and a second connector portion, wherein the second connector portion mounts the disposable delivery tube to the first connector portion, and the first connector portion mounts the second connector portion and the disposable delivery tube to the fluid source.

7. The apparatus according to claim 6 wherein the proximal end of the second connector portion comprises a surface for receiving the sealed blister.

8. The apparatus according to claim 1 wherein the lumen of the nozzle comprises a variable diameter along a longitudinal axis of the lumen.

9. The apparatus according to claim 6 wherein the first connector portion comprises an internal cavity having a cavity thread disposed thereon, the second connector portion comprises an outer surface having a connector thread disposed thereon, and further wherein the cavity thread is configured to mate with the connector thread.

10. The apparatus according to claim 6 wherein the first connector portion comprises an internal cavity comprising at least one L-shaped inset formed therein, wherein the second connector portion comprises an outer surface having at least one extrusion formed thereon, and further wherein the at least one extrusion is sized to be received in the at least one L-shaped inset formed in the internal cavity of the first connector portion, whereby to releasably mount the second connector portion to the first connector portion.

11. The apparatus according to claim 6 wherein the first connector portion comprises an internal cavity comprising at least one connection clip extending inboard thereof, wherein the second connector portion comprises an outer surface having at least one connector insert groove formed therein for receiving the at least one connection clip, whereby to releasably mount the second connector portion to the first connector portion.

12. The apparatus according to claim 6 wherein the apparatus further comprises a snap clamp for connecting the second connector portion to the first connector portion.

13. The apparatus according to claim 12 wherein the first connector portion comprises an internal cavity, wherein the second connector portion comprises a proximal flange and a distal flange, wherein the proximal flange of the second connector portion is sized to be received in the internal cavity of the first connector portion, and further wherein when the proximal flange of the second connector portion is disposed in the internal cavity of the first connector portion, the snap clamp is disposed over the distal flange and the first connector portion so as to releasably mount the second connector portion to the first connector portion.

14. The apparatus according to claim 1 wherein the nozzle comprises an inner wall defining the lumen, an outer wall disposed radially outboard of the inner wall and a chamber extending between the inner wall and the outer wall.

15. The apparatus according to claim 14 wherein the outer wall comprises at least one exhaust port for exhausting excess fluid out of the chamber, the at least one exhaust port further comprising a filter.

16. The apparatus according to claim 15 wherein the filter captures the entrained powdered agent exiting the at least one exhaust port.

17. The apparatus according to claim 14 wherein the chamber comprises at least one baffle.

18. The apparatus according to claim 1 wherein the lumen of the nozzle comprises at least one exhaust port formed in a sidewall defining the lumen for exhausting excess fluid out of the lumen.

19. The apparatus according to claim 1 further comprising a suction cup distal interface disposed over the distal end of the nozzle.

20. An apparatus for transdermal delivery of a powdered agent to a patient, the apparatus comprising:
a fluid source comprising a fluid;
a nozzle extending distally from the fluid source, the nozzle comprising a proximal end, a distal end and a lumen extending from the proximal end to the distal end;
a seat disposed at the proximal end of the nozzle;
a sealed blister with a concave dome proximal surface and a flat distal surface containing the powdered agent positioned on the seat of the nozzle disposed within the lumen;
a blade disposed distal to the seat with the lumen, the blade comprising a blade element configured to pierce the sealed blister when the sealed blister is deformed by passage of the fluid through the sealed blister, the blade element comprising at least one selected from the group consisting of a triangle blade, a flat straight blade, an arching blade, a wire cross blade, a flat cross blade, a pointed cross blade, and a flat pyramid blade; and
an actuation element for releasing the fluid from the fluid source, wherein actuation causes the released fluid to push the sealed blister forward, deforming the concave dome of the sealed blister to move distally toward the blade, thereby causing the blade to pierce the sealed blister to release the powdered agent into the stream of fluid for entrainment and delivery through the distal end of the nozzle.
